(19) United States
(12) Patent Application Publication
(10) Pub. No.: US 2023/0219065 A1
(43) Pub. Date: Jul. 13, 2023

(54) TITANIUM OXIDE COMPOSITION, DISPERSION LIQUID, AND MEMBER HAVING TITANIUM OXIDE COMPOSITION IN SURFACE LAYER

(71) Applicant: Shin-Etsu Chemical Co., Ltd., Tokyo (JP)

(72) Inventors: Mikiya HINOUE, Kamisu-shi (JP); Tomohiro INOUE, Kamisu-shi (JP); Manabu FURUDATE, Kamisu-shi (JP)

(73) Assignee: Shin-Etsu Chemical Co., Ltd., Tokyo (JP)

(21) Appl. No.: 18/010,847

(22) PCT Filed: Jun. 4, 2021
(86) PCT No.: PCT/JP2021/021355

§ 371 Date: Dec. 16, 2022

(30) Foreign Application Priority Data
Jun. 19, 2020 (JP) 2020-106170

(51) Int. Cl. B01J 21/06; B01J 21/16; B01J 21/08; B01J 29/06; B01J 35/00; B01J 37/04; B01J 37/02; B01J 37/00; A61L 9/014

(52) U.S. Cl. CPC B01J 21/063; B01J 21/16; B01J 21/08; B01J 29/06; B01J 35/0013; B01J 35/006; B01J 37/04; B01J 37/0215; B01J 37/00; A61L 9/014

(57) ABSTRACT

Provided is a titanium oxide composition that has a high capability to decompose odor-causing substances, is less likely to cause re-emission of an odor-causing substance(s) due to adsorption of water, and exhibits an excellent particle dispersion stability. The titanium oxide composition contains titanium oxide particles, a component A and a component B. The component A is at least one kind selected from a group of sepiolite and attapulgite, and the component B is at least one kind selected from a group of high silica zeolite and hydrophobic silica. A mass ratio of the component A to the titanium oxide particles is 0.75 to 3.25, and a mass ratio of the component B to the component A is 0.25 to 3.0. Also provided is a member having such titanium oxide composition on its surface.

### TECHNICAL FIELD

The present invention relates to a titanium oxide composition for adsorbing and decomposing odor-causing substances; a dispersion liquid containing such composition; and a member having the titanium oxide composition in a surface layer.

### BACKGROUND ART

In recent years, due to the consumers' heightened awareness of health, “safety and security” in living spaces are now demanded in addition to comfortableness, whereby materials having a deodorizing effect are now demanded for the purpose of suppressing hazardous volatile organic compounds (VOC: Volatile Organic Compounds) that are emitted from livingware and buildings; and suppressing unpleasant odors that are closely associated with human life, such as the smell of sweat, the aging odor, the smell of cigarette, and the smell of food waste.

As a deodorizing method performed with the aid of a deodorant, there are for example a chemical deodorizing method and a physical deodorizing method which are employed selectively depending on intended purposes. The chemical deodorizing method is to achieve an odorless state by subjecting an odor-causing substance(s) to a chemical reaction with deodorizing components, and is thus capable of carrying out deodorization with respect to a particular odor-causing substance at a high selectivity. The physical deodorizing method is to eliminate an odor-causing substance(s) from the air via physical adsorption, and is capable of relatively easily and simultaneously adsorbing multiple odor-causing substances with one adsorbent. As such adsorbent, there are used activated carbon, zeolite, silica gel, alumina, titania, cyclodextrin and the like. However, deodorization by an adsorbent requires replacement of such adsorbent as an adsorption capability will be lost once an adsorption equilibrium has been reached due to an odor-causing substance(s) or the like.

A deodorizing method performed with the aid of a photocatalyst utilizes a mechanism attributed to the decomposition of an odor-causing substance(s), whereby the catalyst itself remains unchanged, and a fresh surface can thus be retained all the time, which makes it unnecessary to supplement the catalyst. In view of these merits, studies have been actively conducted in recent years on decomposing odor-causing substances with a photocatalyst. However, since a photocatalyst usually has a low adsorption capability, the deodorizing speed thereof is slow, and decomposition cannot take place efficiently if the concentration of odor-causing substances is low. Thus, there is a method for replenishing the adsorption capability of a photocatalyst by combining a photocatalyst and an adsorbent.

In the inventions disclosed in Patent documents 1 and 2, sepiolite, silica and the like are used as adsorbents. However, adsorbents with hydrophilic surfaces, such as sepiolite and silica are known to have a strong affinity for water, adsorb water in the air, and thus allow an odor-causing substance(s) that have once adsorbed thereto to be re-emitted.

In the inventions disclosed in Patent documents 3 and 4, high silica zeolite that is hydrophobic is used as an adsorbent to prevent an odor-causing substance(s) from being re-emitted. However, a hydrophobic adsorbent surface leads to a low adsorption capability to adsorb a gas having hydrophilic groups. Further, high silica zeolite usually has a low dispersity in water, and there will thus be exhibited a low dispersion stability of the particles in an aqueous dispersion liquid, whereby it is difficult to form a uniform composition, and the strength of such composition may thus be impaired.

In the invention disclosed in Patent document 5, while there is used an activated carbon having a superior adsorption capability with respect to a number of odor-causing substances, the activated carbon as an adsorbent absorbs lights required for a photocatalytic function be exerted, and thereby hinders the decomposition of an odor-causing substance(s) by a photocatalyst; an activated carbon is thus not fit to be combined with a photocatalyst.

### PRIOR ART DOCUMENTS

### Patent Documents

Patent document 1: JP-A-2001-259003
Patent document 2: JP-A-2002-136811
Patent document 3: JP-A-2008-272651
Patent document 4: JP-A-2018-158316
Patent document 5: JP-A-2003-225572

### SUMMARY OF THE INVENTION

### Problems to be Solved by the Invention

It is an object of the present invention to provide a titanium oxide composition that has a high capability to decompose odor-causing substances, is less likely to cause re-emission of an odor-causing substance(s) due to adsorption of water, and exhibits an excellent particle dispersion stability; and a member having such titanium oxide composition on a surface.

### Means to Solve the Problems

The inventors of the present invention diligently conducted a series of studies to achieve the above object, and completed the invention as follows. That is, the inventors found that a composition containing three kinds of particles which are titanium oxide particles, particles of a component A (e.g. sepiolite) and particles of a component B (e.g. high silica zeolite) at given ratios exhibited a high capability to decompose odor-causing substances, and was able to suppress re-emission of an odor-causing substance(s) due to adsorption of water; and that a dispersion liquid thereof exhibited an excellent particle dispersion stability.

[1]
A titanium oxide composition containing:
   titanium oxide particles;
   a component A that is at least one kind selected from a group of sepiolite and attapulgite; and
   a component B that is at least one kind selected from a group of high silica zeolite and hydrophobic silica,
wherein a mass ratio of the component A to the titanium oxide particles is 0.75 to 3.25, and a mass ratio of the component B to the component A is 0.25 to 3.0.
   [2]
The titanium oxide composition according to [1], wherein the component A is sepiolite, and the component B is high silica zeolite.
   [3]
The titanium oxide composition according to [1] or [2], wherein the titanium oxide particles have an average particle size of 5 to 30 nm.
   [4]
The titanium oxide composition according to any one of [1] to [3], wherein the titanium oxide composition further contains an aqueous dispersion medium.
   [5]
A member having the titanium oxide composition according to any one of [1] to [3] on a surface.

### Effects of the Invention

The composition of the present invention contains three kinds of particles which are titanium oxide particles, particles of a component A (e.g. sepiolite) and particles of a component B (e.g. high silica zeolite) at given ratios, exhibits a higher-than-ever capability to decompose odor-causing substances, and suppresses re-emission of an odor-causing substance(s) due to adsorption of water.

Further, since a dispersion liquid of the present invention contains the three kinds of particles which are the titanium oxide particles, the particles of the component A (e.g. sepiolite) and the particles of the component B (e.g. high silica zeolite) at given ratios, and has an excellent particle dispersion stability, a superior workability will be achieved at the time of coating, and deterioration in strength of the composition will be suppressed as well.

Thus, a member having the titanium oxide composition of the present invention on its surface can bring about an effect of, for example, suppressing hazardous volatile organic compounds (VOC) that are emitted from livingware and buildings; and suppressing unpleasant odors that are closely associated with human life, such as the smell of sweat, the aging odor, the smell of cigarette, and the smell of food waste.

### MODE FOR CARRYING OUT THE INVENTION

The present invention is described in detail hereunder.

### <Titanium Oxide Particles>

As a crystal phase of titanium oxide particles, there are generally known three of them which are the rutile-type, anatase-type and brookite-type, of which it is preferred that those of the anatase-type or rutile-type be mainly used. Here, the expression “mainly” refers to occupying normally not less than 50% by mass, preferably not less than 70% by mass, more preferably not less than 90% by mass, or oven 100% by mass of the whole titanium oxide particle crystals.

As the titanium oxide particles, in order to improve their odor-causing substance decomposition performance, there may be used those with a metal compound such as a compound of platinum, gold, silver, palladium, iron, copper, zinc, nickel or the like being supported on titanium oxide particles; those doped with an element such as tin, nitrogen, sulfur, carbon or a transition metal; or even a titanium oxide for photocatalyst.

It is more preferable if a titanium oxide for photocatalyst is used, because there can be achieved a stronger odor-causing substance decomposition performance when irradiated with a light.

The titanium oxide for photocatalyst is a general photocatalytic titanium oxide, and is preferably a visible light responsive photocatalytic titanium oxide designed to respond to a visible light of 400 to 800 nm.

Here, examples of an odor-causing substance in this specification include odorous components such as ammonia, acetic acid, hydrogen sulfide, methyl mercaptan, trimethylamine, formaldehyde, acetaldehyde, toluene, ethyl acetate, ethylene, benzene, acetone, pyridine, isovaleric acid, nonenal and indole.

The titanium oxide particles preferably have a 50% cumulative distribution diameter D₅₀ (possibly referred to as “average particle size” hereunder) of 5 to 30 nm, more preferably 5 to 20 nm, on volumetric basis when measured by a dynamic light scattering method using a laser light. This is because when the average particle size is smaller than 5 nm, an insufficient deodorizing performance may be exhibited; and when the average particle size is larger than 30 nm, a dispersion liquid with the titanium oxide particles being dispersed in an aqueous dispersion medium may be opaque. Here, as a device for measuring the average particle size, there may be used, for example, ELSZ-2000ZS (by Otsuka Electronics Co., Ltd.), NANOTRAC UPA-EX150 (by Nikkiso Co., Ltd.) or LA-910 (by HORIBA, Ltd.).

### <Component A>

A component A is at least one kind selected from a group of sepiolite and attapulgite; one kind thereof may be used alone, or multiple kinds thereof may be used in combination.

Sepiolite is a clay mineral having many activated hydroxyl groups on a surface comprised of an aqueous magnesium silicate. Unlike a two-dimensional crystal structure such as that of talc, sepiolite has a 2:1 ribbon structure which is a three-dimensional chain structure. In the present invention, other than sepiolite, there may also be used attapulgite (magnesium-aluminum silicate (Mg, Al)₂Si₄O₁₀(OH)₆H₂O as a chain-structured salt; CAS registry number: 12174-11-7) which is a hormite-based clay mineral and has a structure analogous to that of sepiolite; preferably, sepiolite is used.

In terms of adsorption and decomposition rate of an odor-causing substance, it is preferred that the specific surface area of the component A be 100 to 1,000 m²/g, more preferably 120 to 500 m²/g. Further, there are no restrictions on the shape of the component A; other than a fibrous one, there may also be employed any of a block-like and granular ones. Here, the specific surface area is a value measured by a gas adsorption method.

### <Component B>

A component B is at least one kind selected from a group of high silica zeolite and hydrophobic silica; one kind thereof may be used alone, or multiple kinds thereof may be used in combination.

The composition of high silica zeolite is such that it is a crystalline aqueous aluminosilicate containing silica at a ratio higher than that of alumina, and cavities inside the crystal of high silica zeolite is hydrophobic. In general, a zeolite with a SiO₂/Al₂O₃ ratio of 10 or higher in terms of weight is called high silica zeolite. The general formula of high silica zeolite is expressed as A_{2/n}O·Al₂O_{3z}·xSiO₂·yH₂O (A=metal cation such as Na, Ca and K, n=atomic valence); a significant hydrophilicity will be exhibited when the SiO₂/Al₂O₃ ratio of the zeolite framework is low, whereas a significant hydrophobicity will be exhibited when such SiO₂/Al₂O₃ ratio is high. Since a borderline thereof is usually said to be about 30, it is preferred that the high silica zeolite used as the component B have a SiO₂/Al₂O₃ ratio of 30 to 80. Here, the SiO₂/Al₂O₃ ratio can be calculated by a quantitative analysis of element via an inductively coupled plasma emission spectroscopic analysis (ICP-AES).

As the component B, there may also be used a high silica zeolite with the replaceable hydrogen ions therein being replaced with metal ions such as copper ions. In the present invention, as the component B, there may also be used, for example, a hydrophobic silica with a hydrophobic adsorbent surface as is the case with high silica zeolite; preferably, high silica zeolite is used.

Hydrophobic silica is a silicon oxide compound that has been subjected to a hydrophobization treatment using a trimethylsilylation agent. It is preferred that the hydrophobic silica used as the component B have a hydrophobicity of not lower than 20; the higher the hydrophobicity is, the more favorable it is, and there are no particular limitations on an upper limit thereof.

Here, the hydrophobicity in this specification refers to a concentration expressed by a methanol volume % at which a powder to be treated starts to swell in a mixed solution of water and methanol, and is measured under the following conditions.

### <Method for Measuring Hydrophobicity>

Specifically, 50 mL of a pure water is put into a 200 mL beaker, followed by adding 0.2 g of a sample thereto and then stirring them with a magnetic stirrer. The tip end of a burette filled with methanol is put into the solution to deliver methanol by drops while performing stirring; the hydrophobicity is then obtained by the following formula, provided that Y mL represents a methanol additive amount required for the sample to be completely dispersed in the water.

Hydrophobicity M={Y/(50+Y)}×100

Further, it is preferred that the component B have an average particle size of not larger than 50 μm, more preferably not larger than 20 μm, even more preferably not larger than 5 μm. This is because when the average particle size is larger than 50 μm, not only the dispersion stability of the particles in the dispersion liquid will be impaired, but there will also thus be a concern that the strength of a composition may deteriorate. Here, the average particle size is measured by a dynamic light scattering method using a laser light.

### <Titanium Oxide Composition>

A titanium oxide composition of the present invention is such a composition that it contains the titanium oxide particles and the components A and B, and that a mass ratio of the component A to the titanium oxide particles is 0.75 to 3.25, and a mass ratio of the component B to the component A is 0.25 to 3.0.

It is not preferable if the mass ratio of the component A to the titanium oxide particles is smaller than 0.75 or larger than 3.25. This is because when this mass ratio is smaller than 0.75, there cannot be achieved a sufficient adsorption effect with respect to an odor-causing substance, whereby the decomposition rate of such odor-causing substance will decrease; and when this mass ratio is larger than 3.25, not only the decomposition rate of an odor-causing substance will decrease as a light progression required for titanium oxide to exert a photocatalytic activity is hindered, but a re-emission of the odor-causing substance owing to adsorption of water will also be unignorable. Further, it is not preferable if the mass ratio of the component B to the component A is smaller than 0.25 or larger than 3.0. This is because when this mass ratio is smaller than 0.25, there cannot be achieved a sufficient adsorption effect with respect to an odor-causing substance, whereby the decomposition rate of such odor-causing substance will decrease; and when this mass ratio is larger than 3.0, the decomposition rate of an odor-causing substance will decrease as a light progression required for titanium oxide to exert a photocatalytic activity is hindered. In terms of adsorption effect with respect to an odor-causing substance and improvement in the decomposition rate of an odor-causing substance, it is more preferred that the mass ratio of the component B to the component Abe 1.25 to 2.5.

### <Titanium Oxide Dispersion Liquid>

As one embodiment of the titanium oxide composition of the present invention, there may also be listed a titanium oxide composition further containing an aqueous dispersion medium in addition to the titanium oxide particles (i.e. titanium oxide dispersion liquid), the component A and the component B. Since the titanium oxide dispersion liquid of the present invention is superior in dispersion stability of particles, a uniform surface layer of titanium oxide can be formed if applied to a later-described member. Thus, it is preferred that the composition be used as a titanium oxide dispersion liquid.

As the aqueous dispersion medium of the titanium oxide dispersion liquid, an aqueous solvent is normally used, and it is preferred that water be used. However, there may also be used a water-soluble organic solvent mixable with water, or a mixed solvent prepared by mixing water and a water-soluble organic solvent at any ratio. As water, preferred are, for example, a deionized water, a distilled water and a pure water. Further, as the water-soluble organic solvent, preferred are, for example, alcohols such as methanol, ethanol and isopropanol; glycols such as ethylene glycol and propylene glycol; and glycol ethers such as ethylene glycol monomethyl ether, ethylene glycol monoethyl ether and propylene glycol-n-propyl ether. There may be used one kind of these aqueous dispersion media, or two or more kinds of them in a combined manner. If using a mixed solvent, it is preferred that the water-soluble organic solvent be contained in the mixed solvent at a ratio of larger than 0% by mass, but not larger than 50% by mass, more preferably at a ratio of not larger than 20% by mass, even more preferably at a ratio of not larger than 10% by mass.

The titanium oxide dispersion liquid of the present invention is such a dispersion liquid that it contains the titanium oxide particles and the components A and B, and that the mass ratio of the component A to the titanium oxide particles is 0.75 to 3.25, and the mass ratio of the component B to the component A is 0.25 to 3.0.

It is not preferable if the mass ratio of the component A to the titanium oxide particles is smaller than 0.75 or larger than 3.25. This is because when this mass ratio is smaller than 0.75, the dispersion liquid will exhibit a poor dispersion stability, which leads to a concern that the strength of the composition may deteriorate; and when this mass ratio is larger than 3.25, the composition will exhibit a decreased decomposition rate of an odor-causing substance. Further, it is not preferable if the mass ratio of the component B to the component A is smaller than 0.25 or larger than 3.0. This is because when this mass ratio is smaller than 0.25, the composition will exhibit a decreased decomposition rate of an odor-causing substance; and when this mass ratio is larger than 3.0, the dispersion liquid will exhibit a poor dispersion stability of the particles therein, which leads to a concern that the strength of the composition may deteriorate. In terms of adsorption effect with respect to an odor-causing substance, improvement in the decomposition rate of an odor-causing substance, and dispersion stability of the particles in the dispersion liquid, it is more preferred that the mass ratio of the component B to the component Abe 1.25 to 2.5.

In terms of ease in producing a later-described titanium oxide composition (surface layer) with a given thickness, it is preferred that the concentration of the titanium oxide particles in the titanium oxide dispersion liquid be 0.01 to 30% by mass, particularly preferably 0.5 to 20% by mass.

There are no particular limitations on a method for producing the titanium oxide composition of the present invention so long as the composition is produced by mixing the titanium oxide particles and the components A and B at the abovementioned ratios.

Further, in the case of the titanium oxide composition further containing the aqueous dispersion medium, the composition may be produced by mixing the titanium oxide particle dispersion liquid and the components A and B at the abovementioned ratios. Although not particularly limited, it is preferred that the production method of the aqueous dispersion medium-containing titanium oxide composition be such that a titanium oxide precursor aqueous solution of a titanium peroxide or the like is at first subjected to crystal growth via a hydrothermal treatment, followed by adding the components A and B to the titanium oxide particle dispersion liquid obtained.

### <Member Having Surface Layer Containing Titanium Oxide>

The titanium oxide dispersion liquid can be used for the purpose of forming, on the surface of a member, a composition having a property to decompose odor-causing substances. The member(s) may have various shapes depending on their intended purposes and uses.

Here, the member in this specification may for example be an interior architectural material such as a wall material for buildings, a wallpaper, a ceiling material, a floor material, a tile, a brick, a wooden board, a resin board, a metallic plate, a tatami and a bathroom material; a vehicle interior material such as a wall material for automobiles, trains or the like, a ceiling material, a floor material, a seat, a hand rail and a hanging strap; furniture and livingware such as a curtain, a blind, a rug, a partition board, a glass, a mirror, a film, a desk, a chair, a bed and a storage rack; and a home electric appliance such as a deodorization filter, an air purifier, an air conditioner, a refrigerator, a laundry machine, a personal computer, a printer, a tablet, a touch panel and a telephone, of which a deodorization filter or the like is suitable.

Here, the material of the member may for example be an organic material or an inorganic material.

Examples of the organic material include synthetic resin materials such as polyvinyl chloride resin (PVC), polyethylene (PE), polypropylene (PP), polycarbonate (PC), an acrylic resin, polyacetal, a fluorocarbon resin, a silicone resin, an ethylene-vinyl acetate copolymer (EVA), an acrylonitrile-butadiene rubber (NBR), polyethylene terephthalate (PET), polyethylene naphthalate (PEN), polyvinyl butyral (PVB), an ethylene-vinyl alcohol copolymer (EVOH), a polyimide resin, polyphenylene sulfide (PPS), polyetherimide (PEI), polyetheretherimide (PEEI), polyetheretherketone (PEEK), a polyamide resin (PA), a melamine resin, a phenolic resin and an acrylonitrile-butadiene-styrene (ABS) resin; natural materials such as a natural rubber; and semisynthetic materials of the abovelisted synthetic resin materials and natural materials. It is possible that these materials have already been processed into products having given shapes and structures, such as a film, sheet, fiber material, fiber product and other molded products as well as laminates.

Examples of the inorganic material include non-metallic inorganic materials and metallic inorganic materials.

Examples of the non-metallic inorganic materials include glass, ceramics, stone materials and plasters. It is possible that these materials have already been processed into products having various shapes, such as tiles, glass, mirrors, walls, filters and decorative materials.

Examples of the metallic inorganic materials include a cast iron, steel, iron, iron alloy, stainless steel, aluminum, aluminum alloy, aluminum nitride, zirconia, silicon carbide, silicon nitride, a clay mineral, alumina, titania, silica, nickel, nickel alloy and zinc die-cast. They may be plated with any of the above metal inorganic materials or coated with any of the above organic materials, or may be used to plate the surfaces of the above organic materials or non-metallic inorganic materials.

As a method for forming the titanium oxide composition on the surface of the member, there may be employed, for example, a method where the titanium oxide dispersion liquid is to be applied to the surface of the member by a method such as spray coating, flow coating, dip coating, spin coating, Meyer bar coating, reverse roll coating, gravure coating, knife coating, kiss coating or die coating, followed by performing, for example, drying and/or film transfer.

A drying temperature after the application may be variously determined depending on the target member to be coated; the drying temperature is preferably 0 to 1,000° C., more preferably 10 to 800° C., even more preferably 20 to 700° C. This is because if the drying temperature is lower than 0° C., the dispersion liquid and/or coating liquid may freeze and thus become unusable. Further, since the crystal water of sepiolite or zeolite will be perfectly dehydrated at a temperature of not lower than 600° C., sintering at a temperature higher than 700° C. is not only uneconomical, but may also cause the strength of the composition to be impaired easily.

A drying time after the application may be appropriately selected depending on the application method and drying temperature; the drying time is preferably 10 min to 72 hours, more preferably 20 min to 48 hours. This is because it is not preferable if the drying time is shorter than 10 min or longer than three days. If the drying time is shorter than 10 min, the composition may be retained on the surface of the member in an insufficient manner; and if the drying time is longer than three days, a poor economic efficiency in production will be incurred.

The thickness of the composition (surface layer) on the surface of the member may be appropriately selected; this thickness is preferably 100 nm to 50 μm, more preferably 200 nm to 50 μm, even more preferably 500 nm to 30 μm. This is because if such layer thickness is smaller than 100 nm, there may be exhibited an insufficient property to decompose odor-causing substances; and if such layer thickness is larger than 50 μm, the surface layer may be easily peeled away from the surface of the member.

### WORKING EXAMPLES

The present invention is described in detail hereunder with reference to working and comparative examples; the present invention shall not be limited to the following working examples. Performance tests in the present invention were performed as follows.

(1) 50% Cumulative Distribution Diameter (D₅₀) of Titanium Oxide Dispersion Liquid D₅₀ of the titanium oxide dispersion liquid was calculated as a 50% cumulative distribution diameter on volumetric basis, that was measured by a laser light-aided dynamic light scattering method using a particle size distribution measurement device (ELSZ-2000ZS by Otsuka Electronics Co., Ltd.).

### (2) Acetaldehyde Gas Re-Emission Test

An evaluation sample was put into a 1 L gasbag, followed by injecting into the gasbag 300 mL of an acetaldehyde gas having a concentration of 100 ppm. After leaving the gasbag to stand still for 45 min, it was regarded that an adsorption equilibrium had been reached, whereby the concentration of the acetaldehyde gas was measured. The sample after reaching the adsorption equilibrium was taken out and then put into a new 1 L gasbag, followed by injecting into this gasbag 300 mL of an air having a relative humidity of 100%. After leaving this gasbag to stand still for 15 min, the concentration of the acetaldehyde emitted from the evaluation sample was measured. A ratio of the re-emitted acetaldehyde was calculated by the following formula 1. There, a Tedlar bag with one opening cock (by AS ONE Corporation) was used as the 1 L gasbag, and the concentration of the acetaldehyde gas was measured by an acetaldehyde detector tube 92 L (by GASTEC CORPORATION); evaluation was then conducted based on the criteria shown below.
Favorable (marked “∘”) ⋅ ⋅ ⋅ Ratio of acetaldehyde re-emitted was smaller than 5%.
Slightly unfavorable (marked “Δ”) ⋅ ⋅ ⋅ Ratio of acetaldehyde re-emitted was 5 to 10%.
Unfavorable (marked “x”) ⋅ ⋅ ⋅ Ratio of acetaldehyde re-emitted was greater than 10%.

Ratio of acetaldehyde re-emitted[%]=Concentration of acetaldehyde re-emitted/(Initial concentration−Acetaldehyde concentration at the time of adsorption equilibrium)×100 Formula 1:

### (3) Acetaldehyde Gas Decomposition Performance Evaluation Test

The evaluation sample that had reached the adsorption equilibrium was put into a 1 L gasbag, followed by injecting into this gasbag 700 mL of an acetaldehyde gas of a concentration of 20 ppm to measure an initial concentration of the acetaldehyde gas. After carrying out irradiation with an ultraviolet light of 2.0 (mW/cm²) for 20 min, the concentration of the acetaldehyde gas was again measured. As a light source of the test, there was used a UVLED lamp (product model “HLDL-600X480U6-PSC” by CCS Inc.) A decomposition rate of the odorous gas was calculated by the following formula 2, and was evaluated based on the criteria shown below.
Favorable (marked “∘”) ⋅ ⋅ ⋅ Decomposition rate was greater than 90%.
Slightly unfavorable (marked “Δ”) ⋅ ⋅ ⋅ Decomposition rate was 70 to 90%.
Unfavorable (marked “x”) ⋅ ⋅ ⋅ Decomposition rate was smaller than 70%.

Decomposition rate[%]=(Initial concentration−Residual concentration)/Initial concentration×100 Formula 2:

### (4) Dispersion Stability Evaluation of Titanium Oxide Dispersion Liquid

Measurement of the dispersion stability of the titanium oxide dispersion liquid prepared was performed by a multi-specimen/dispersion stability evaluation/particle distribution measurement device (LUMiSiZER 610 by LUM Japan Co., Ltd.). LUMiSiZER was operated in such a manner that 0.4 mL of the titanium oxide dispersion liquid prepared was put into a cell (each PC 2 mm cell by LUM Japan Co., Ltd.), where cycle parameters were set to 2,500 rpm, 300 profiles, measurement interval 10 sec, 25° C., optical coefficient 1. There was measured a permeability at a measurement position of 115 mm 60 sec after starting centrifugal separation, and the permeability was evaluated based on the criteria shown below.
Favorable (marked “∘”) ⋅ ⋅ ⋅ Permeability at measurement position of 115 mm 60 sec after starting centrifugal separation was 0% to smaller than 50%.
Unfavorable (marked “x”) ⋅ ⋅ ⋅ Permeability at measurement position of 115 mm 60 sec after starting centrifugal separation was not smaller than 50%.

### (5) Comprehensive Evaluation

From the results of the acetaldehyde gas re-emission test, the acetaldehyde gas decomposition performance evaluation test and the dispersion stability evaluation test of titanium oxide dispersion liquid, a comprehensive evaluation was conducted based on the criteria shown below.
Very favorable (marked “Δ”) ⋅ ⋅ ⋅ Total score in all evaluations was 6 points.
Favorable (marked “B”) ⋅ ⋅ ⋅ Total score in all evaluations was 5 points.
Slightly unfavorable (marked “C”) ⋅ ⋅ ⋅ Total score in all evaluations was 3 to 4 points.
Unfavorable (marked “D”) ⋅ ⋅ ⋅ Total score in all evaluations was 0 to 2 points.

Point allocation for the calculation of the total score was ∘⋅ ⋅ ⋅ 2 points, Δ ⋅ ⋅ ⋅ 1 point, x ⋅ ⋅ ⋅ 0 point.

### <Preparation of Titanium Oxide Particle Dispersion Liquid>

### Production Example 1

After diluting a 36% by mass titanium chloride (IV) aqueous solution 10 times with a pure water, a 10% by mass ammonia water was gradually added thereto to neutralize and hydrolyze the solution so as to obtain a precipitate of titanium hydroxide. pH at that time was 8. The precipitate of titanium hydroxide thus obtained was then deionized by repeating addition of pure water and decantation. A 35% by mass hydrogen peroxide water was then added to such deionized titanium hydroxide precipitate so that hydrogen peroxide/titanium hydroxide (molar ratio) would be 10, followed by allowing them to sufficiently react at 60° C. for two hours to obtain a peroxotitanic acid solution (l a) (solid content concentration 1% by mass).

Next, 400 mL of the peroxotitanic acid solution (la) was put into a 500 mL autoclave so as to be subjected to a hydrothermal treatment at 150° C. for 90 min, followed by adding a pure water thereto for the purpose of concentration adjustment, thereby obtaining a dispersion liquid of titanium oxide particles (1A) (solid content concentration 2.0% by mass).

D₅₀ of the titanium oxide particles in the dispersion liquid was 18 nm.

### Working Example 1

### <Preparation of Titanium Oxide Dispersion Liquid>

A titanium oxide dispersion liquid was obtained by mixing and dispersing 20 mL of the titanium oxide particle dispersion liquid (1A), 0.8 g of a sepiolite (PANGEL AD: Si₁₂Mg₈O₃₀(OH)₄(OH₂)₄·8H₂O (CAS63800-37-3) by Kusumoto Chemicals, Ltd.) and 1.0 g of a high silica zeolite (USKY-700 by UNION SHOWA K.K.) The composition of the dispersion liquid is shown in Table 1, and the average particle sizes and specific surface areas of the materials used are shown in Table 2.

### <Production of Evaluation Sample>

A bar coater (No. 7 by Daiichi Rika Co., Ltd.) was used to apply the titanium oxide dispersion liquid to a plasma surface-treated PET film (Lumirror T60 by Toray Industries, Inc.) so that a thickness of 16.0 μm would be formed thereon. The film coated was then dried in an oven of 80° C. for 30 min, followed by cutting off the coated part into a 10 cm squared sample so as to obtain an evaluation sample with a titanium oxide composition being formed on the film.

The evaluation sample thus obtained was then subjected to the acetaldehyde gas re-emission test (Table 3) and the acetaldehyde gas decomposition performance evaluation test (Table 4); the results thereof are shown in each table.

Further, the titanium oxide dispersion liquid was subjected to the dispersion stability evaluation test (Table 5), and the result thereof is shown in the table. Moreover, the result of the comprehensive evaluation is shown in Table 6.

### Working Example 2

Evaluations were conducted in a similar manner as the working example 1, except that the titanium oxide dispersion liquid was obtained by mixing and dispersing 0.4 g of the sepiolite (PANGEL AD by Kusumoto Chemicals, Ltd.) and 0.5 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 3

Evaluations were conducted in a similar manner as the working example 1, except that the titanium oxide dispersion liquid was obtained by mixing and dispersing 1.2 g of the sepiolite (PANGEL AD by Kusumoto Chemicals, Ltd.) and 1.5 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 4

Evaluations were conducted in a similar manner as the working example 1, except that the titanium oxide dispersion liquid was obtained by mixing and dispersing 0.8 g of the sepiolite (PANGEL AD by Kusumoto Chemicals, Ltd.) and 0.4 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 5

Evaluations were conducted in a similar manner as the working example 1, except that the titanium oxide dispersion liquid was obtained by mixing and dispersing 0.8 g of the sepiolite (PANGEL AD by Kusumoto Chemicals, Ltd.) and 2.0 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 6

Evaluations were conducted in a similar manner as the working example 1, except that the titanium oxide dispersion liquid was obtained by mixing and dispersing 0.8 g of a sepiolite (PANSIL by Kusumoto Chemicals, Ltd.) and 2.0 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 7

Evaluations were conducted in a similar manner as the working example 1, except that the titanium oxide dispersion liquid was obtained by mixing and dispersing 0.8 g of an attapulgite (Attagel 40 by BASF) and 2.0 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 8

Evaluations were conducted in a similar manner as the working example 1, except that the titanium oxide dispersion liquid was obtained by mixing and dispersing 0.8 g of the sepiolite (PANGEL AD by Kusumoto Chemicals, Ltd.) and 2.0 g of a hydrophobic silica (HDK (registered trademark) H30 by Wacker Asahikasei Silicone Co., Ltd., M value 52) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 9

Evaluations were conducted in a similar manner as the working example 1, except that the titanium oxide dispersion liquid was obtained by mixing and dispersing 1.2 g of the sepiolite (PANGEL AD by Kusumoto Chemicals, Ltd.) and 3.0 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Working Example 10

Evaluations were conducted in a similar manner as the working example 1, except that the titanium oxide dispersion liquid was obtained by mixing and dispersing 0.4 g of the sepiolite (PANGEL AD by Kusumoto Chemicals, Ltd.) and 0.2 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Comparative Example 1

Evaluations were conducted in a similar manner as the working example 1, except that the titanium oxide dispersion liquid was obtained by mixing and dispersing 0.2 g of the sepiolite (PANGEL AD by Kusumoto Chemicals, Ltd.) and 0.5 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Comparative Example 2

Evaluations were conducted in a similar manner as the working example 1, except that the titanium oxide dispersion liquid was obtained by mixing and dispersing 1.4 g of the sepiolite (PANGEL AD by Kusumoto Chemicals, Ltd.) and 0.7 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Comparative Example 3

Evaluations were conducted in a similar manner as the working example 1, except that the titanium oxide dispersion liquid was obtained by mixing and dispersing 0.8 g of the sepiolite (PANGEL AD by Kusumoto Chemicals, Ltd.) and 0.1 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

### Comparative Example 4

Evaluations were conducted in a similar manner as the working example 1, except that the titanium oxide dispersion liquid was obtained by mixing and dispersing 0.8 g of the sepiolite (PANGEL AD by Kusumoto Chemicals, Ltd.) and 2.8 g of the high silica zeolite (USKY-700 by UNION SHOWA K.K.) into 20 mL of the titanium oxide particle dispersion liquid (1A) (Table 1). The titanium oxide dispersion liquid or evaluation sample obtained was subjected to the acetaldehyde gas re-emission test (Table 3), the acetaldehyde gas decomposition performance evaluation test (Table 4) and the dispersion stability test (Table 5); the results thereof are shown in each table. Moreover, the result of the comprehensive evaluation is shown in Table 6.

TABLE 1

| | Titanium oxide | | | | | | |
|---|---|---|---|---|---|---|---|
| | Titanium | | | | | | |
| | oxide | 1A | | | | | |
| | dispersion | Solid | Component A | | | | |
| | liquid | content | PANGEL | | ATTAGEL | Component B | |
| | 1A | 1B | AD | PANSIL | 40 | USKY-700 | HDK ®H30 |
| | [mL] | [g] | [g] | [g] | [g] | [g] | [g] |
| Working example 1 | 20 | 0.4 | 0.8 | — | — | 1 | — |
| Working example 2 | 20 | 0.4 | 0.4 | — | — | 0.5 | — |
| Working example 3 | 20 | 0.4 | 1.2 | — | — | 1.5 | — |
| Working example 4 | 20 | 0.4 | 0.8 | — | — | 0.4 | — |
| Working example 5 | 20 | 0.4 | 0.8 | — | — | 2 | — |
| Working example 6 | 20 | 0.4 | — | 0.8 | — | 2 | — |
| Working example 7 | 20 | 0.4 | — | — | 0.8 | 2 | — |
| Working example 8 | 20 | 0.4 | 0.8 | — | — | — | 2 |
| Working example 9 | 20 | 0.4 | 1.2 | — | — | 3 | — |
| Working example 10 | 20 | 0.4 | 0.4 | — | — | 0.2 | — |
| Comparative example 1 | 20 | 0.4 | 0.2 | — | — | 0.5 | — |
| Comparative example 2 | 20 | 0.4 | 1.4 | — | — | 0.7 | — |
| Comparative example 3 | 20 | 0.4 | 0.8 | — | — | 0.1 | — |
| Comparative example 4 | 20 | 0.4 | 0.8 | — | — | 2.8 | — |

TABLE 2

| | | | | Specific |
|---|---|---|---|---|
| | | Average particle | | surface |
| | | size | | area |
| Titanium oxide | Titanium oxide | 18 | nm | — |
| | dispersion liquid 1A | | | |
| Component A | PANGEL AD | Not larger than 5 μm | | 320 m²/g |
| | PANSIL | Not larger than 5 μm | | 270 m²/g |
| | ATTAGEL40 | 11 | μm | 150 m²/g |
| Component B | USKY-700 | 3 to 5 | μm | — |

| | | |
|---|---|---|
| HDK ®H30 | Not larger than 40 μm | 300 m²/g |

TABLE 3

| | Component A/ | Component B/ | Ratio of acetaldehyde | Re-emission |
|---|---|---|---|---|
| | Titanium oxide [—] | Component A [—] | re-emitted [%] | evaluation |
| Working example 1 | 2 | 1.25 | 0.0 | ∘ |
| Working example 2 | 1 | 1.25 | 0.0 | ∘ |
| Working example 3 | 3 | 1.25 | 0.0 | ∘ |
| Working example 4 | 2 | 0.5 | 3.2 | ∘ |
| Working example 5 | 2 | 2.5 | 0.0 | ∘ |
| Working example 6 | 2 | 2.5 | 0.0 | ∘ |
| Working example 7 | 2 | 2.5 | 0.0 | ∘ |
| Working example 8 | 2 | 2.5 | 0.8 | ∘ |
| Working example 9 | 3 | 2.5 | 0.0 | ∘ |
| Working example 10 | 1 | 0.5 | 2.6 | ∘ |
| Comparative example 1 | 0.5 | 2.5 | 1.2 | ∘ |
| Comparative example 2 | 3.5 | 0.5 | 13.2 | x |
| Comparative example 3 | 2 | 0.125 | 18.6 | x |
| Comparative example 4 | 2 | 3.5 | 0 | ∘ |

TABLE 4

| | | | | Decomposition |
|---|---|---|---|---|
| | Component A/ | Component B/ | Decomposition | performance |
| | Titanium oxide [—] | Component A [—] | rate [%] | evaluation |
| Working example 1 | 2 | 1.25 | 95.2 | ∘ |
| Working example 2 | 1 | 1.25 | 87.6 | Δ |
| Working example 3 | 3 | 1.25 | 96.8 | ∘ |
| Working example 4 | 2 | 0.5 | 93.2 | ∘ |
| Working example 5 | 2 | 2.5 | 97.7 | ∘ |
| Working example 6 | 2 | 2.5 | 97.2 | ∘ |
| Working example 7 | 2 | 2.5 | 82.8 | Δ |
| Working example 8 | 2 | 2.5 | 84.6 | Δ |
| Working example 9 | 3 | 2.5 | 98.2 | ∘ |
| Working example 10 | 1 | 0.5 | 82.6 | Δ |
| Comparative example 1 | 0.5 | 2.5 | 76.4 | Δ |
| Comparative example 2 | 3.5 | 0.5 | 86.6 | Δ |
| Comparative example 3 | 2 | 0.125 | 61.5 | x |
| Comparative example 4 | 2 | 3.5 | 97.4 | ∘ |

TABLE 5

| | Cmponent A/ | Component B/ | Permeability | Dispersibility |
|---|---|---|---|---|
| | Titanium oxide [—] | Component A [—] | [%] | evaluation |
| Working example 1 | 2 | 1.25 | 24 | ∘ |
| Working example 2 | 1 | 1.25 | 32 | ∘ |
| Working example 3 | 3 | 1.25 | 18 | ∘ |
| Working example 4 | 2 | 0.5 | 22 | ∘ |
| Working example 5 | 2 | 2.5 | 30 | ∘ |
| Working example 6 | 2 | 2.5 | 32 | ∘ |
| Working example 7 | 2 | 2.5 | 34 | ∘ |
| Working example 8 | 2 | 2.5 | 36 | ∘ |
| Working example 9 | 3 | 2.5 | 26 | ∘ |
| Working example 10 | 1 | 0.5 | 30 | ∘ |
| Comparative example 1 | 0.5 | 2.5 | 64 | x |
| Comparative example 2 | 3.5 | 0.5 | 16 | ∘ |
| Comparative example 3 | 2 | 0.125 | 18 | ∘ |
| Comparative example 4 | 2 | 3.5 | 58 | x |

TABLE 6

| | | Decomposition | | | |
|---|---|---|---|---|---|
| | Re-emission | performance | Dispersibility | | |
| | evaluation | evaluation | evaluation | Total score | Grade |
| Working example 1 | ∘ | ∘ | ∘ | 6 | A |
| Working example 2 | ∘ | Δ | ∘ | 5 | B |
| Working example 3 | ∘ | ∘ | ∘ | 6 | A |
| Working example 4 | ∘ | ∘ | ∘ | 6 | A |
| Working example 5 | ∘ | ∘ | ∘ | 6 | A |
| Working example 6 | ∘ | ∘ | ∘ | 6 | A |
| Working example 7 | ∘ | Δ | ∘ | 5 | B |
| Working example 8 | ∘ | Δ | ∘ | 5 | B |
| Working example 9 | ∘ | ∘ | ∘ | 6 | A |
| Working example 10 | ∘ | Δ | ∘ | 5 | B |
| Comparative example 1 | ∘ | Δ | x | 3 | C |
| Comparative example 2 | x | Δ | ∘ | 3 | C |
| Comparative example 3 | x | x | ∘ | 2 | D |
| Comparative example 4 | ∘ | ∘ | x | 4 | C |

As can be seen from the working examples 1 to 10, in the cases of the titanium oxide compositions in which the mass ratio of the component A to the titanium oxide particles was 0.75 to 3.25, and the mass ratio of the component B to the component A was 0.25 to 3.0, there was exhibited an excellent property to decompose odor-causing substances, an odor-causing substance that had once adsorbed thereto was not re-emitted easily, and the dispersion liquid exhibited an excellent dispersion stability of the particles.

As can be seen from the working example 6, even when using a sepiolite with a different specific surface area, there was exhibited an excellent property to decompose odor-causing substances, an odor-causing substance that had once adsorbed to the composition was not re-emitted easily, and the dispersion liquid exhibited an excellent dispersion stability of the particles.

As can be seen from the working example 7, even when using an attapulgite, there was exhibited an excellent property to decompose odor-causing substances, an odor-causing substance that had once adsorbed to the composition was not re-emitted easily, and the dispersion liquid exhibited an excellent dispersion stability of the particles.

As can be seen from the working example 8, even when using a hydrophobic silica, there was exhibited an excellent property to decompose odor-causing substances, an odor-causing substance that had once adsorbed to the composition was not re-emitted easily, and the dispersion liquid exhibited an excellent dispersion stability of the particles.

As can be seen from the comparative example 1, when the amount of the component A in the composition was small, the effects brought about by the adsorbent were small such that there was exhibited a low property to decompose odor-causing substances, and the dispersion liquid also exhibited a poor dispersion stability of the particles.

As can be seen from the comparative example 2, when the amount of the component A in the composition was excess, acetaldehyde that had adsorbed to the component A was re-emitted easily.

As can be seen from the comparative example 3, when the amount of the component B in the composition was small, an adsorbability to acetaldehyde was weak such that there was exhibited a low property to decompose acetaldehyde, and there was obtained a large ratio of the acetaldehyde that had once adsorbed to the composition but was re-emitted.

As can be seen from the comparative example 4, when the amount of the component B in the composition was excess, the dispersion stability of the particles in the dispersion liquid deteriorated.

The above results indicate that the composition of the present invention is capable of efficiently decomposing odor-causing substances, and suppressing re-emission of odor-causing substances, where the dispersion liquid thereof has an excellent dispersion stability of the particles.

1. A titanium oxide composition comprising:
titanium oxide particles;
a component A that is at least one kind selected from a group of sepiolite and attapulgite; and
a component B that is at least one kind selected from a group of high silica zeolite and hydrophobic silica,
wherein a mass ratio of the component A to the titanium oxide particles is 0.75 to 3.25, and a mass ratio of the component B to the component A is 0.25 to 3.0.

2. The titanium oxide composition according to claim 1, wherein the component A is sepiolite, and the component B is high silica zeolite.

3. The titanium oxide composition according to claim 1, wherein the titanium oxide particles have an average particle size of 5 to 30 nm.

4. The titanium oxide composition according to claim 1, wherein the titanium oxide composition further comprises an aqueous dispersion medium.

5. A member having the titanium oxide composition according to claim 1 on a surface.
